(19) United States
(12) Patent Application Publication
(10) Pub. No.: US 2025/0272888 A1
(43) Pub. Date: Aug. 28, 2025

(54) RECONSTRUCTION METHOD, DATA PROCESSING UNIT AND MEDICAL IMAGING DEVICE

(71) Applicant: Siemens Healthineers AG, Forchheim (DE)

(72) Inventor: Alois Regensburger, Poxdorf (DE)

(21) Appl. No.: 19/059,936

(22) Filed: Feb. 21, 2025

(30) Foreign Application Priority Data
Feb. 22, 2024 (DE) 10 2024 201 615.2

(51) Int. Cl. G06T 11/00; G06T 17/00

(52) U.S. Cl. CPC G06T 11/003; G06T 17/00

(57) ABSTRACT

For a simple 3D reconstruction, a method for reconstructing a 3D volume of a moving object under examination is provided, having the following steps: providing a dataset containing a multiplicity of projection images, which was generated during a rotation run of an acquisition system of an X-ray device about the object under examination; selecting at least one first projection image from the dataset; providing a generalized 3D model of the object under examination; generating a personalized 3D model of the object under examination from the generalized 3D model using the first projection image; selecting a second projection image from the dataset; registering the second projection image to the personalized 3D model of the object under examination; and reconstructing a 3D volume of the object under examination on the basis of the first and at least one further registered projection image.

### CROSS REFERENCE TO RELATED APPLICATIONS

This application claims the benefit DE 10 2024 201 615.2 filed on Feb. 22,2024, which is hereby incorporated by reference in its entirety.

FIELD

Embodiments relate to reconstructing a 3D volume of a moving object under examination.

### BACKGROUND

In order to obtain 3D volume images of an object under examination, i.e. of a patient or of a body part or organ of a patient, a multiplicity of projection images are generated during rotation of an X-ray acquisition system about the object under examination. What are known as cone-beam CT (CBCT) or DynaCT acquisitions may then be reconstructed into a 3D volume image. A rotation run of this type has a relatively long acquisition time, which means that movements of the patient, for instance caused by respiratory movement, heartbeat and involuntary movement, are a major problem in particular for depictions of the spinal column, of the internal organs and for neurological acquisitions, that severely impairs the quality of the imaging.

Motion compensation or motion correction is often performed in order to cancel out the patient movements. In general, this is done by performing an initial image reconstruction using known reconstruction approaches, followed by image-based motion compensation via backprojection or re-registration. However, this approach often fails as a result of excessive patient movements during the acquisition process. In particular, the initialization of the motion-compensated image reconstruction is difficult when patient movements are strong.

The website https://www.cs.cornell.edu/projects/bigsfm/ discloses an approach that uses machine learning methods to create a 3D model on the basis of a multiplicity of acquisitions of an object or a building (solving the global SfM translations problem).

The article by Amirhossein Saedpanah et al, “Geometrical Self-Calibration of CBCT Systems”, 12th Conference on Industrial Computed Tomography, Fürth, Germany (iCT 2023) discloses performing self-calibrations of CBCT acquisitions, which self-calibrations relate to an object.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a method for reconstructing a moving object under examination that allows stable motion compensation even in the case of strong movements.

The method for reconstructing a 3D volume of a moving object under examination includes the following steps: providing a dataset containing a multiplicity of projection images, that was generated during a rotation run of an acquisition system of an X-ray device about the object under examination; selecting at least one first projection image from the dataset; providing a generalized 3D model of the object under examination; generating a personalized 3D model of the object under examination from the generalized 3D model using the first projection image; selecting a second projection image from the dataset; registering the second projection image to the personalized 3D model of the object under examination; and reconstructing a 3D volume of the object under examination on the basis of the first and at least one further registered projection image, i.e. in particular the first, the second and possibly further projection images.

Embodiments are focused primarily on stabilizing the initialization of a 3D reconstruction. For this purpose, it is not absolutely necessary for the initial motion correction to be very precise. By using a generalized 3D model of the object under examination, that is then personalized by a first projection image, it is possible to create a basis for further optimizations that ensures the fundamental success of a reconstruction of the 3D volume of the object under examination.

In the article by Karthik Shetty, Annette Birkhold, et al, “HOOREX: Higher Order Optimizers for 3D Recovery from X-Ray Images”, Workshop on Machine Learning for Multimodal Healthcare Data at conference, Saturday, July 29, Hawaii, USA, a 3D digital twin model of vertebrae of a patient is generated using a single 2D projection image of the vertebrae of the patient. It involves an adaptive approach in which a neural network is trained to generate a skeleton model by fusing a regression-based method with an optimization-based method. First, a regression method is used as the prior knowledge learned by a network, because such a method may be deployed easily. Then, the corresponding reprojection error is optimized iteratively therefrom for a better 2D match.

Embodiments use a similar approach with a generalized 3D model and a first projection image, from which a personalized 3D model is generated, for instance by a trained function, as a first draft for a reconstruction, and then to correct the movements on further projection images by registering at least one or more further projection images. The projection images that have been motion-corrected by registration may subsequently be reconstructed into a 3D volume image. It is thereby possible to guarantee a stable reconstruction method, even when there are strong movements of the object under examination, that may then be optimized. This provides improved diagnosis and treatment for the patient.

The method is suitable not just for moving objects under examination but also for poorly or inadequately calibrated acquisition trajectories of the X-ray system in the rotation about the object under examination. The method allows improved reconstruction even when there is insufficiently accurate knowledge about the rotation of an X-ray system about the patient.

According to an embodiment, the following additional steps are performed before the reconstruction step: updating the personalized 3D model of the object under examination using the second projection image; and selecting at least one further projection image from the dataset; and registering the at least one further projection image to the updated personalized 3D model of the object under examination. The reconstruction may then be performed on the basis of the at least three projection images used. The personalized 3D model is hence further refined (personalized) using the already registered second projection image or, in some cases, further projection images, so that further optimizations and quality improvements of the 3D volume may be achieved on the basis of at least three projection images.

According to an embodiment, before the reconstruction step, the steps of updating the personalized 3D model, selecting a further projection image and registering the further projection image are repeated until a termination criterion. The use of a further iterative method until a termination criterion allows automation of the quality improvement. A termination criterion may be selected as required, for example selected such that enough projection images are motion-corrected to perform a high-quality reconstruction, but not more than necessary, so that the computing effort may be kept low at the same time.

According to an embodiment, the termination criterion is formed by a specified threshold regarding the number of projection images or by a registration parameter. The number of projection images (or a certain percentage of acquired projection images) may be specified, for example such that the image quality of the reconstructed 3D volume reaches a certain quality level. A registration parameter may mean, for example, that all projection images that exceed or do not reach a predetermined measure of movement are used, and other projection images are not used. It may also be provided to use only projection images having angulations (projection angles) that are sufficiently spaced apart.

According to an embodiment, the generalized 3D model is formed by a shape model. Shape models are fundamentally known, for example the statistical shape model (SSM) or the statistical shape intensity model (SSIM) or Skinned Multi-Person Linear model (SMPL), and therefore may be used to apply the method easily. For example, shape models of bone structures are known from the article by Shetty, K., Birkhold, A., Jaganathan, S., Strobel, N., Egger, B., Kowarschik, M., Maier, A.: “Boss: Bones, organs and skin shape model”. Computers in Biology and Medicine, Volume 165, Article 107383, October 2023.

According to an embodiment, all the projection images are successively selected and registered. This allows a full and optimum reconstruction to be performed using the entire dataset of projection images.

According to an embodiment, the selection of the projection images is made randomly or on the basis of an algorithm. An algorithm may specify which boundary conditions must be fulfilled. For example, the selection may be made on the basis of which projection image is most similar to the 3D model or which differs the most from the 3D model. It is also possible to select, for example, projection images having projection angles that differ by a certain angle, or, for example, every 2nd, 5th or 10th projection image.

According to an embodiment, before the reconstruction, the already registered projection images are re-registered to the current (latest) personalized 3D model. This allows a further improvement in the motion correction of the already registered projection images and hence even better quality of the 3D volume to be reconstructed. In addition, all the remaining, still unused, projection images may also be registered to the current personalized 3D model, and then a reconstruction may be performed on the basis of all the projection images.

According to an embodiment, the personalized 3D model is generated by applying at least one pre-trained machine learning function, and/or the updating of the personalized 3D model is performed by applying at least one pre-trained machine learning function. In particular, the pre-trained function is generated by fusing a regression-based method with an optimization-based method.

Embodiments further provide a data processing unit for performing an above-described method, and a medical imaging device having a data processing unit, wherein the medical imaging device has an acquisition system for acquiring a dataset containing a multiplicity of projection images during a rotation run about the object under examination.

### BRIEF DESCRIPTION OF THE FIGURES

FIG. 1 depicts a series of steps of a method for reconstructing a 3D volume of a moving object under examination according to an embodiment.

FIG. 2 depicts a further series of steps of a method for reconstructing a 3D volume of a moving object under examination according to an embodiment.

FIG. 3 depicts a further series of steps of a method for reconstructing a 3D volume of a moving object under examination according to an embodiment.

FIG. 4 depicts a medical X-ray device for performing the method according to an embodiment.

### DETAILED DESCRIPTION

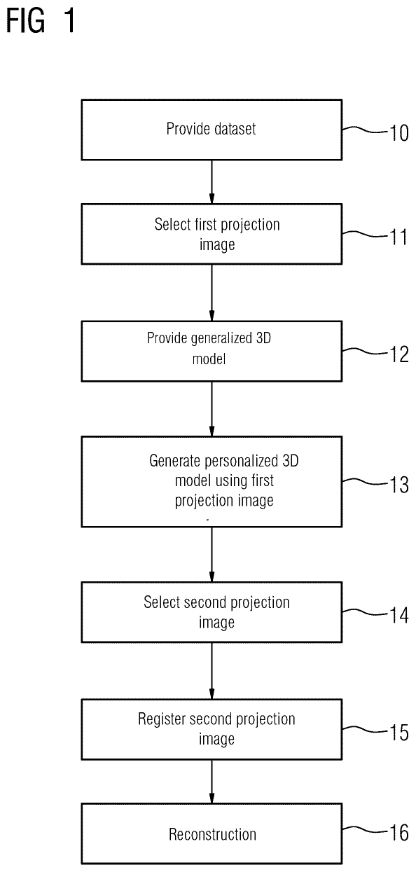

When reconstructing 3D volumes from a dataset containing a multiplicity of projection images acquired during a rotation run of an acquisition system about an object under examination, problems caused by patient or organ movements arise in many cases. Especially when movements are large, this may lead to a reconstruction that generates a 3D volume of unacceptable quality or even to reconstruction not being feasible at all. The present method for reconstructing a 3D volume of a moving object under examination rectifies this problem by using a 3D model that is first personalized by a single projection image. This makes motion correction of further projection images possible and hence guarantees a stable reconstruction. FIG. 1 depicts the steps of the method.

In a first step 10 a dataset containing a multiplicity of projection images, that were acquired from different projection directions with respect to the object under examination, is initially provided. The providing may be done, for example, by retrieving from a memory or database a dataset already acquired previously, or by acquiring a dataset directly by an X-ray device. For example, the dataset may be a CBCT acquisition (cone-beam CT) under a rotation trajectory. In this case, projection images are generally acquired over an angle range of at least 180° about the object under examination. The X-ray device may be a C-arm X-ray device, for example.

In a second step 11, a first projection image is selected from the dataset. The selection of the first projection image may be random or made according to an algorithm. For example, the first projection image may be the projection image acquired first or a projection image from a first, predefined projection direction. It may also be a projection image of particularly high image quality, of a certain contrast, having a certain image content or having another defined property.

In a third step 12 is provided a generalized 3D model of the object under

examination (for instance the entire patient, a segment, a body part or an organ of the patient). For example, this may be a shape model, for instance a statistical shape model (SSM) or a statistical shape intensity model (SSIM), or a BOSS or OSSA in the case of bones (see earlier).

In a fourth step 13, a personalized 3D model of the object under examination is generated from the generalized 3D model, with the first projection image being used for this purpose. An already pre-trained machine learning function may be used for this, for example. For instance, the method disclosed in the article “HOOREX: Higher Order Optimizers for 3D Recovery from X-Ray Images” (citation above) or a similar method may be used for this.

In a fifth step 14, a second projection image of the dataset is then selected. The selection of the first projection image may likewise be random or made according to an algorithm. The projection angle of the second projection image preferably differs significantly (>5° or better >10°) from the projection angle of the first projection image in order to make a meaningful later 3D reconstruction possible.

In a sixth step 15, the second projection image is registered to the personalized 3D model of the object under examination. A known method for 2D/3D registration may be used for this purpose, for instance by machine learning, as in Unberath, Mathias, et al. “The impact of machine learning on 2D/3D registration for image-guided interventions: A systematic review and perspective” Frontiers in Robotics and AI 8 (2021): 716007, or by an algorithm such as in Y. Lei and Y. Zhang, “An improved 2D-3D medical image registration algorithm based on modified mutual information and expanded Powell method,” 2013 IEEE International Conference on Medical Imaging Physics and Engineering, Shenyang, China, 2013, pp. 24-29.

Subsequently, in a seventh step 16, a 3D volume is reconstructed from the registered projection images, so in the present example from the first and second projection images, since these have now undergone motion correction as a result of the registration.

In the simplest case, that is shown in FIG. 1, thus only two projection images are used for a reconstruction. In general, however, even more projection images are selected, since the image quality is considerably higher for a 3D reconstruction with three or, better still, a multiplicity of projection images. In the simplest case again, the further selected projection images are likewise registered to the personalized 3D model, and reconstructed into a 3D volume after the registration of all (of the ones selected or even of all in total).

A further refinement of the method may be implemented by performing, after the registration of the second projection image and also each further projection image, an update of the personalized 3D model using the second projection image and also each further projection image. This may again be performed in the same way as in the original personalization of the general 3D model, for instance using the above-described HOOREX method and a trained function.

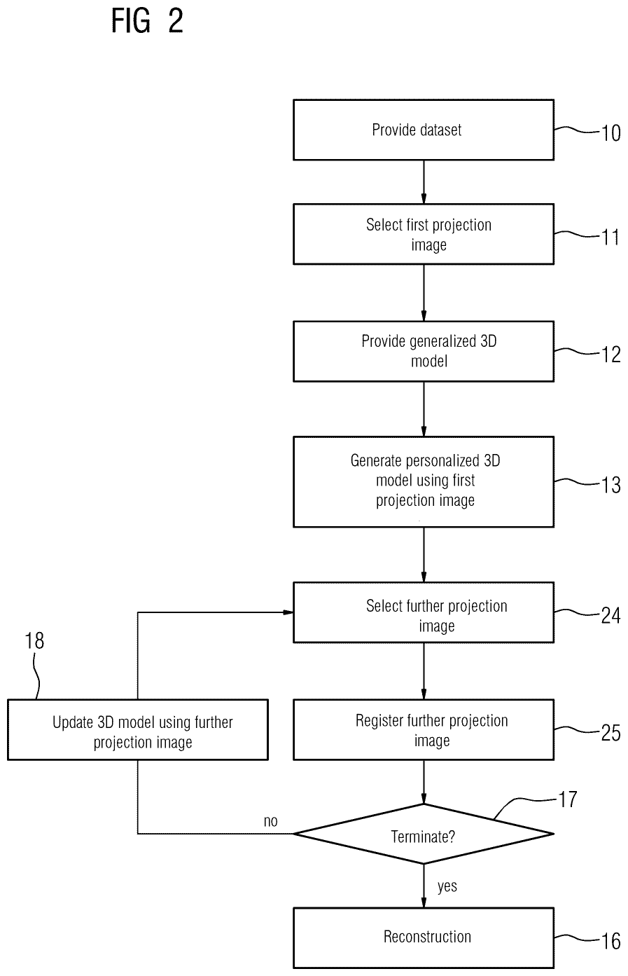

FIG. 2 depicts the steps of a further method. The first four steps 10 to 13 are initially identical to the steps of FIG. 1. In an eighth step 24, a further projection image is then selected. The selection of the further projection image may likewise be random or made according to an algorithm. In a ninth step 25, the further projection image is then registered to the personalized 3D model of the object under examination. Once again, this may be done using a known 2D/3D registration method. Then, in a tenth step 17, a termination criterion (or a plurality of termination criteria) is interrogated. The termination criterion may be, for example, a specified threshold regarding the number of projection images, so for instance if a certain number or a certain percentage of projection images have already been used. It may also be provided to check the image quality, and to terminate after using those projection images that satisfy a certain quality criterion (for instance with regard to image sharpness, contrast, motion etc.). The termination criterion may also include a registration parameter, for instance a predetermined measure for reprojection errors or convergence of the registration algorithm. If the termination criterion (or termination criteria) is not satisfied, then in an eleventh step 18, the personalized 3D model is updated using the further projection image (for instance as above by the described method and a trained function) in order to then return again to the eighth step 24, the selection of a further projection image. This loop is repeated until a termination criterion is satisfied. If a termination criterion is satisfied, the seventh step 16 is performed, a reconstruction of all previously registered projection images into a 3D volume.

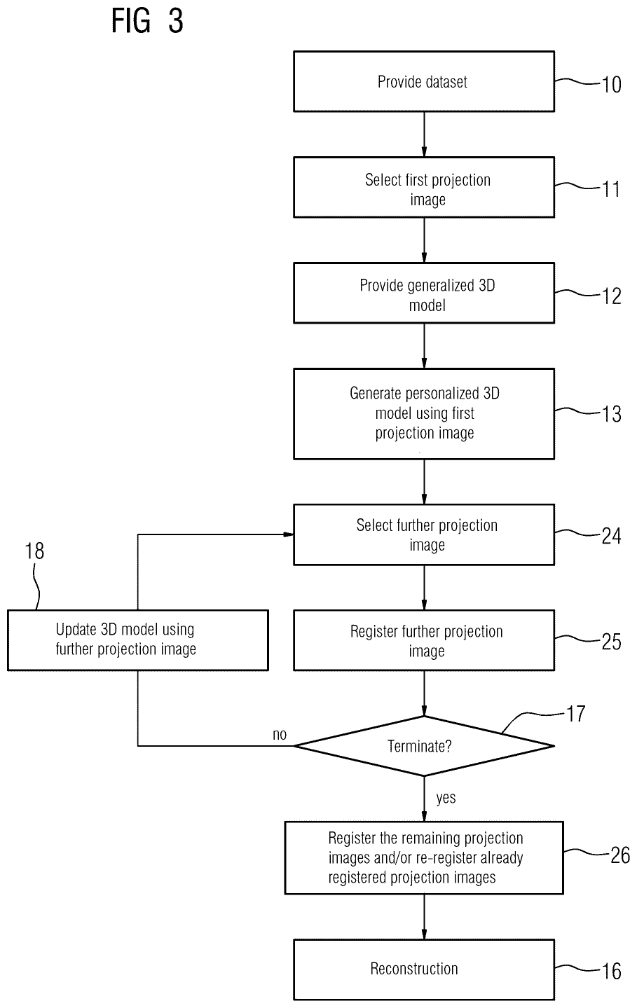

FIG. 3 depicts a further method, in which in a twelfth step 26, before the reconstruction, a registration of the remaining, still unused, projection images to the current personalized 3D model is performed. Alternatively or additionally, the already previously registered projection images may also be re-registered to the current personalized 3D model.

In addition, further methods for motion correction may also be performed, whether before the start of the method or before the particular reconstruction.

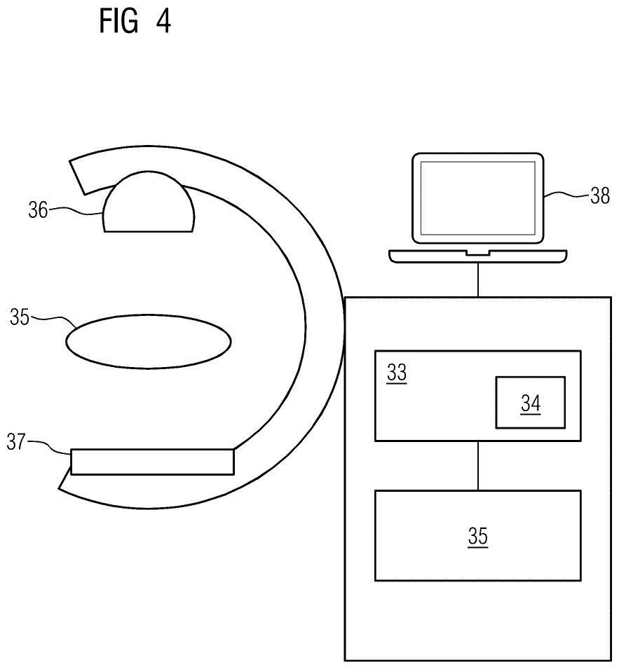

FIG. 4 depicts a medical X-ray device 30, that is controlled by a system controller 33 and has a data processing unit 31 for performing the above-described steps of the method. The X-ray device 30 has an acquisition system, for example a C-arm 32 having an X-ray source 36 and an X-ray detector 37, that is designed to generate under a rotation trajectory a multiplicity of projection images, for instance for a CBCT acquisition, of an object 35 under examination. The data processing unit 31 is designed to select projection images, to generate a personalized 3D model from a generalized 3D model using at least one projection image, for instance using trained functions, to register projection images to the personalized 3D model, and to perform reconstructions.

As an alternative to an acquisition, a dataset already acquired previously may be provided, for instance by a provider unit, and used. A data storage medium 34 may be present for storing a dataset, interim and final results of the method, or the generalized or personalized 3D model.

Overall, an approach is shown that allows, despite large movements of a patient, initial (and not necessarily precise) motion correction of the individual projections. Subsequently, a first corrected image reconstruction may then be performed, on the basis of which, further known methods may then be carried out for more accurate motion compensation. Even in the case of 3D acquisitions containing very strong movements that may be modeled only poorly, this method may be used for initialization, and then other methods from the prior art may be used iteratively for refinement.

Embodiments provide particularly good image quality in 3D volume images impaired by patient movements by using a method for reconstructing a 3D volume of a moving object under examination is provided, including the following steps: providing a dataset containing a multiplicity of projection images, that was generated during a rotation run of an acquisition system of an X-ray device about the object under examination; selecting at least one first projection image from the dataset; providing a generalized 3D model of the object under examination; generating a personalized 3D model of the object under examination from the generalized 3D model using the first projection image; selecting a second projection image from the dataset; registering the second projection image to the personalized 3D model of the object under examination; and reconstructing a 3D volume of the object under examination on the basis of the registered projection images.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description. CLAIMS

1. A method for reconstructing a 3D volume of a moving object under examination, the method comprising:
providing a dataset containing a multiplicity of projection images, the dataset generated during a rotation run of an acquisition system of an X-ray device about the object under examination;
selecting at least one first projection image from the dataset;
providing a generalized 3D model of the object under examination;
generating a personalized 3D model of the object under examination from the generalized 3D model using the first projection image;
selecting a second projection image from the dataset;
registering the second projection image to the personalized 3D model of the object under examination; and
reconstructing a 3D volume of the object under examination on a basis of the first projection image and at least one further registered projection image.

2. The method of claim 1, before reconstructing, further comprising:
updating the personalized 3D model of the object under examination using the second projection image;
selecting at least one further projection image from the dataset; and
registering the at least one further projection image to the updated personalized 3D model of the object under examination.

3. The method of claim 2, wherein updating the personalized 3D model, selecting a further projection image, and registering the further projection image are repeated until a termination criterion.

4. The method of claim 1, wherein the generalized 3D model is formed by a shape model.

5. The method of claim 1, wherein all the projection images are successively selected and registered.

6. The method of claim 1, wherein the selection of the second and/or further projection images is random or is made on a basis of an algorithm.

7. The method of claim 1, wherein the selection of the second and/or further projection images is made on a basis of which projection image is most similar to the 3D model or which differs the most from the 3D model.

8. The method of claim 3, wherein the termination criterion is formed by a specified threshold regarding a number of already registered projection images or by a registration parameter.

9. The method of claim 1, wherein, before the reconstruction, already registered projection images are re-registered to a current personalized 3D model.

10. The method of claim 1, wherein the personalized 3D model is generated by applying at least one pre-trained machine learning function, and/or updating of the personalized 3D model is performed by applying at least one pre-trained machine learning function.

11. The method of claim 10, wherein the pre-trained function is generated by fusing a regression-based method with an optimization-based method.

12. A medical imaging device comprising:
an acquisition system for acquiring a dataset containing a multiplicity of projection images during a rotation run about an object under examination; and
a data processing unit configured to:
select at least one first projection image from the dataset;
provide a generalized 3D model of the object under examination;
generate a personalized 3D model of the object under examination from the generalized 3D model using the first projection image;
select a second projection image from the dataset;
register the second projection image to the personalized 3D model of the object under examination; and
reconstruct a 3D volume of the object under examination on a basis of the first projection image and at least one further registered projection image.

13. The system of claim 12, wherein the data processing unit is further configured to, before reconstructing:
update the personalized 3D model of the object under examination using the second projection image;
select at least one further projection image from the dataset; and
register the at least one further projection image to the updated personalized 3D model of the object under examination.

14. The system of claim 13, wherein updating the personalized 3D model, selecting a further projection image, and registering the further projection image are repeated until a termination criterion.

15. The system of claim 12, wherein the generalized 3D model is formed by a shape model.

16. The system of claim 12, wherein all the projection images are successively selected and registered.

17. The system of claim 12, wherein the selection of the second and/or further projection images is random or is made on a basis of an algorithm.

18. The system of claim 12, wherein the selection of the second and/or further projection images is made on a basis of which projection image is most similar to the 3D model or which differs the most from the 3D model.

19. The system of claim 14, wherein the termination criterion is formed by a specified threshold regarding a number of already registered projection images or by a registration parameter.

20. The system of claim 12, wherein, before the reconstruction, the data processing unit is further configured to re-register already registered projection images to the current personalized 3D model.
